(19) United States
(12) Patent Application Publication
(10) Pub. No.: US 2017/0043237 A1
(43) Pub. Date: Feb. 16, 2017

(54) PROTECTIVE MOUTH GUARDS AND RELATED METHODS

(71) Applicant: Steven D. JENSEN, West Jordan, UT (US)

(72) Inventor: Steven D. Jensen, West Jordan, UT (US)

(21) Appl. No.: 15/305,619

(22) PCT Filed: Apr. 21, 2015
(86) PCT No.: PCT/US15/26802

§ 371 Date: Oct. 20, 2016

Related U.S. Application Data
(60) Provisional application No. 61/982,636, filed on Apr. 22, 2014.

(51) Int. Cl. A63B 71/08; A61F 5/56; A61C 5/14

(52) U.S. Cl. CPC A63B 71/085; A61C 5/14; A61F 5/566; A61F 2005/563; A63B 2209/14

(57) ABSTRACT

A laminated mouth guard may include a shape memory layer, one or more of a cushion layer and a puncture resistant layer, and an adhesive layer. The adhesive layer may be gelatinous and may include a polymer. For example, the adhesive layer may include one or more of PVP, polyethyloxazoline, carboxypolymethylene, guar gum, and a hydrogel. In addition to the adhesive layer, the laminated mouth guard may include a sheet material comprising at least one of LDPE, HDPE, LDPP, HDPP, polyurethane, PVA, cotton, silicone rubber and non-woven PE.

### CROSS-REFERENCES TO RELATED APPLICATIONS

This application claims the benefit of PCT Application No. PCT/US2015/026802, filed Apr. 21, 2015, which is a non-provisional of U.S. Provisional Application No. 61/982,636 filed on Apr. 22, 2014, which is hereby incorporated by reference in its entirety.

### TECHNICAL FIELD OF THE INVENTION

The present invention relates to protective mouth guards and related methods. More specifically, the present invention relates to laminate mouth guards made from a plurality of layers.

### SUMMARY OF THE INVENTION

Laminated mouth guard devices, methods of making such mouth guard devices, and methods of using such mouth guard devices are described herein.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific example embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings.

FIG. 1 depicts and exploded view of a laminate mouth guard according to an embodiment of the present invention.

FIG. 2 depicts a top view of a set of upper and lower laminate mouth guards according to an embodiment of the present invention.

### DETAILED DESCRIPTION

There are many conditions and circumstances where a protective mouth guard may be desired in order to protect and preserve the integrity and longevity of one's teeth. Those individuals that engage in sports activities wear mouth guards to protect their teeth from accidental or incidental bumps or collisions they might receive.

There are many individuals that grind their teeth. Among those that grind their teeth, are those that suffer from a handicap and constantly grind their teeth and there are others that brux at night during sleep. If left untreated the teeth eventually become ground flat.

Most mouth guards currently available on the market today are either boil and bite trays or custom trays made by a dentist. Boil and bite trays are a one-size-fits-all generic tray that is softened in hot water prior to a patients bite registration, after biting into the softened tray the tray is removed and allowed to cool into a more permanent form. Boil and bite trays are not form fitting and easily fall from the teeth, therefore the patient must constantly hold their teeth together for functionality. Custom trays made by a dentist are far superior because the tray is made from a precise impression, whereby a tough elastomer is vacuumed-formed over a cast model of the impression. Custom trays are time consuming to make and therefore are prohibitively expensive. Both custom trays and boil and bite trays are designed to be worn multiple times, this requires the patient to be meticulous at cleaning the trays everyday or else the trays become smelly and messy.

In view of the foregoing, it would be desirable to have a custom mouth guard that is inexpensive and disposable. Embodiments of the present invention comprise strips of monolithic and/or other various materials that are adhered together into a tough, flexible, flat laminated strip, one exposed surface of the laminated strip receiving an adhesive layer that is designed to adhere the laminated strip onto the teeth. The laminated strip may be placed initially on the facial surfaces of the teeth then any remainder folded onto the lingual surfaces of the teeth.

The laminate layers may build into the mouth guard the best mix of physical characteristics such as shape-memory, toughness, and abrasion/puncture resistance. In some embodiments, the mouth guard may include one or more of the following physical features to improve its effectiveness:

Abrasion and Puncture Resistant: The laminate mouth guard may include one or more layers with sufficient strength such that it resists perforation during heavy grinding or biting events.

Cushion: The laminate mouth guard may include one or more layers that cushion any impact between the upper and lower arch.

Shape Memory: The laminate mouth guard may include one or more layers that allow the laminated strip to stay folded when placed on the teeth with minimal elastic rebound. The laminated strip will tend to stay in the shape of any direction it is bent.

A sticky adhesive layer: The laminate mouth guard may include an outer layer comprised of a sticky adhesive designed to adhere the laminated mouth guard onto the teeth for the duration of day and/or night. The preferred adhesive is a sticky gelatinous layer similar in rheological properties as a confection known as gummy worms.

Various sheet materials may be utilized in the construction of a laminated mouth guard; these sheet materials may have more than one advantageous physical attribute and are interchangeable as well. A few example embodiments of sheet materials include but are not limited to: LDPE, HDPE, LDPP, HDPP, polyurethane, Parafilm, PVA, PolyVinyl alcohol, cotton fabric, woven cotton, silicone rubber, fabric, Non-woven PE fabric, and any other useful sheet materials.

The adhesive layer may comprise a sticky adhesive that is capable of adhering the laminated strip to the teeth. In some embodiments, the adhesive layer may comprise one or more polymers that become adhesives when formed into hydrogels or gels, such as PVP, polyethyloxazoline, carboxypolymethylene, guar gum, hydrogels derived from cellulose, polyvinyl alcohol, and any other useful adhesive compound. In one embodiment, the adhesive may comprise a polymer such polyethyloxazoline that is blended with water to form a hydrogel. If desired various beneficial compounds can be added to the hydrogels such as flavoring agents, sweeteners, humectants, and other useful compounds. The coalescent hydrogel can also be further dried into a non-coalescent flexible layer similar in rheological properties as a confection known as gummy worms.

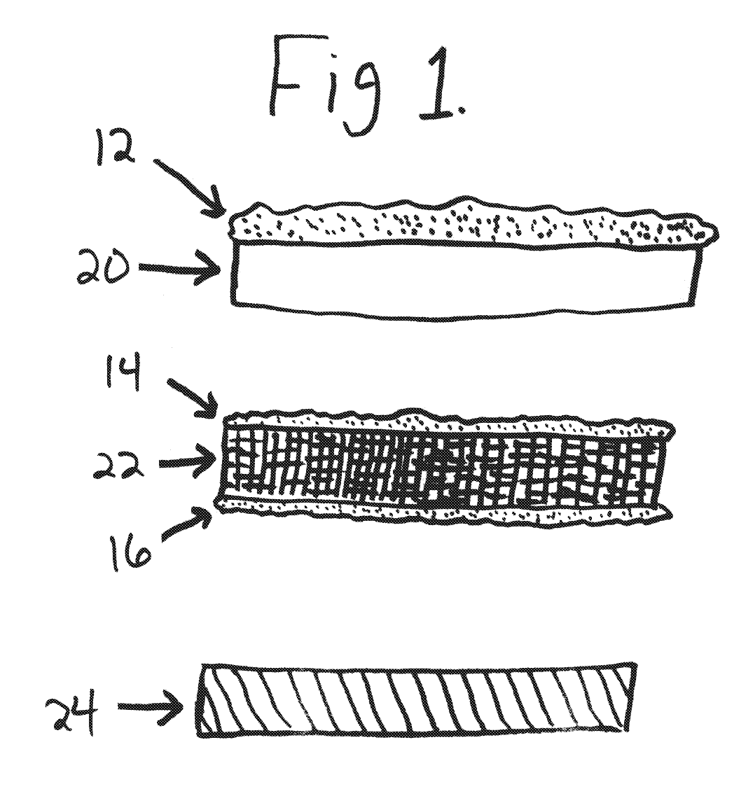

FIG. 1 shows an exploded view of layers of a laminated strip that may be used to form a laminated mouth guard according to an embodiment of the present invention. The multiple layers, when pressed together, form a laminated mouth guard 10 in the form of a laminated strip. The adhesive layer 12 is designed to adhere the laminated mouth guard 10 to the teeth. Adhesive layer 12 can be a coalescent gel or a non-coalescent gelatinous layer. The adhesive used for adhesive layer 12 can also be used as a general purpose adhesive 14, 16 to adhere the various laminate layers together, which it is being used for this purpose in this embodiment. Layer 20 may be Parafilm that is incorporated as a cushion and aid in the shape memory of the laminated mouth guard 10. Layer 22 may be a tough non-woven polypropylene fabric that acts as a cushion and a puncture resistant layer. Layer 24, the outermost layer, may be a sheet of flexible LDPE designed to be puncture and abrasion resistant; the LDPE also forms a water insoluble barrier that protects the adhesive layers 12, 14, 16 from the dilution effects of saliva.

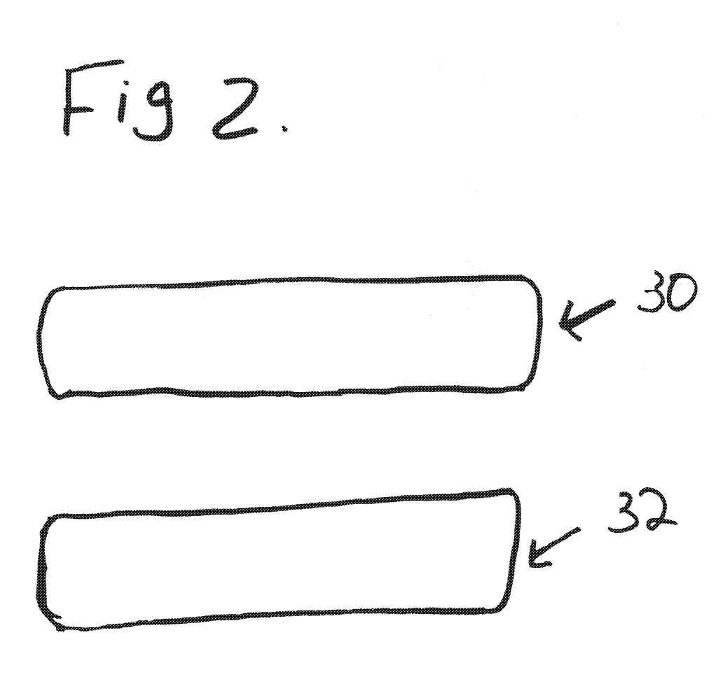

As shown in FIG. 2, lamintated layers may be utilized to manufacture an upper laminated mouth guard strip 30 for fitting to a users upper teeth, and a lower laminated mouth guard strip 32 for fitting to a users lower teeth.

The above examples are given to demonstrate functional examples of laminated mouth guard strips that are designed to protect the teeth during events that would be harmful to the teeth. The laminated mouth guard strip is packaged such that the customer need only open the package and remove the laminated mouth guard strip prior to placement. The end-user simply lines the laminated mouth guard strip with respect to the gingival line of the facial teeth and presses one portion of the laminated mouth guard strip against the facial teeth surfaces, once the facial portion of the strip is adhered to the teeth, then the remainder portion of the strip is folded onto the back or lingual side of the teeth and pressed into place to form a mouth guard. The adhesive strength being sufficient to hold the laminated strip firmly onto the teeth, such that it resists being removed by forces encountered within the oral cavity. Removal of the laminated mouth guard strip ultimately requires the fingers to slowly peel the strip starting at an edge or corner. Once removed the inexpensive laminated mouth guard strip is disposable.

It can be easily seen by the foregoing example that by practice of the present invention many types of laminated mouth guard strips can be constructed, each with customizable characteristics or physical properties as desired.

The present invention may be embodied in other specific forms without departing therefrom. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A laminated mouth guard, comprising:
a shape memory layer;
at least one of a cushion layer and a puncture resistant layer; and
an adhesive layer;

2. The laminated mouth guard of claim 1, wherein the adhesive layer is a gelatinous layer.

3. The laminated mouth guard of claim 2, wherein the adhesive layer comprises a polymer.

4. The laminated mouth guard of claim 2, wherein the adhesive layer comprises at least one of PVP, polyethyloxazoline, carboxypolymethylene, guar gum, and a hydrogel.

5. The laminated mouth guard of claim 2, wherein the adhesive layer further comprises a flavoring agent.

6. The laminated mouth guard of claim 1, comprising a sheet material comprising at least one of LDPE, HDPE, LDPP, HDPP, polyurethane, PVA, cotton, silicone rubber and non-woven PE.
